(19) United States
(12) Patent Application Publication
(10) Pub. No.: US 2024/0005483 A1
(43) Pub. Date: Jan. 4, 2024

(54) METHOD OF TRAINING A MACHINE LEARNING MODEL, METHOD OF ASSESSING ULTRASOUND MEASUREMENT DATA, METHOD OF DETERMINING INFORMATION ABOUT AN ANATOMICAL FEATURE, ULTRASOUND SYSTEM

(71) Applicant: Oxford University Innovation Limited, Oxford (GB)

(72) Inventors: Julia Alison NOBLE, Oxford (Oxfordshire) (GB); Yuan GAO, Oxford (Oxfordshire) (GB); Aris PAPAGEORGHIOU, Oxford (Oxfordshire) (GB)

(21) Appl. No.: 18/027,490

(22) PCT Filed: Sep. 22, 2021
(86) PCT No.: PCT/EP2021/076060

§ 371 Date: Mar. 21, 2023

(30) Foreign Application Priority Data
Sep. 29, 2020 (EP) 20390003.0
Nov. 5, 2020 (GB) 2017510.5

(51) Int. Cl. G06T 7/00; G06T 7/62

(52) U.S. Cl. CPC G06T 7/0012; A61B 8/0866; G06T 7/62

(57) ABSTRACT

Methods of training a machine learning model and using the trained model to assess ultrasound measurement data are disclosed. In one arrangement, training data comprises a plurality of classified frames of ultrasound measurement data. Each of at least a subset of the classified frames is classified as representing an imaging plane capable of providing information about a respective target anatomical feature. First and second samples of frames are selected. A machine learning model derives prototype feature vectors from the first sample and feature vectors from the second sample. A loss function depending on metrics representing distances between the feature vectors and the prototype feature vectors is optimized to train the machine learning model.

The present disclosure relates to assessing ultrasound measurement data using a machine learning model.

It is well known to use ultrasound to obtain information about structures inside the human or animal body. The information may include quantitative measurements for diagnostic use. For example, fetal brain biometry measurements to assess fetal growth can be performed, such as estimation of the head circumference (HC) and transcerebellar diameter (TCD). Such measurements may be performed by manipulating an ultrasound probe until an optimal ultrasound imaging plane is observed by a user of the probe. The parameter of interest is then obtained from the optimal imaging plane. This visual assessment of planes is time consuming, subjective and requires significant training to do well. The approach presents particular challenges where ultrasound image quality is sub-optimal, such as where portable and/or low-cost ultrasound probes are used.

It is an object of the invention to at least partially address one or more of the problems with the prior art discussed above and/or other problems.

According to an aspect of the invention, there is provided a computer-implemented method of training a machine learning model to assess ultrasound measurement data, the method comprising: (a) receiving training data comprising a plurality of classified frames of ultrasound measurement data, each of at least a subset of the classified frames being classified as representing an imaging plane capable of providing information about a respective target anatomical feature corresponding to the classified frame class; (b) selecting from the plurality of classified frames a first sample of frames and a second sample of frames; (c) using the machine learning model to derive a prototype feature vector for each of one or more target anatomical features, each prototype feature vector being derived from feature vectors obtained by inputting to the machine learning model frames from the first sample that belong to a classified frame class corresponding to a respective one of the target anatomical features; (d) using the machine learning model to derive a feature vector for each of the frames in the second sample; (e) calculating metrics representing respective distances, in an embedded space of the feature vectors, between each of the feature vectors derived in (d) and each of the prototype feature vectors derived in (c); and (f) iteratively modifying parameters of the machine learning model and repeating (b)-(e) to optimize a loss function that is a function of the metrics calculated in (e).

The method provides a trained machine learning model that is lightweight and computationally efficient. The model can assess frames of ultrasound data representing different imaging planes, and output numerical values (quality metrics) that indicate how suitable each frame is for extracting information of interest. The model can be implemented on modest computational hardware (e.g. on a mobile device such as a tablet or smart phone) and provide near-real-time feedback to an operator. The method is demonstrated to be effective even where relatively inexpensive ultrasound hardware is used. The approach can therefore be deployed in a wider range of settings than alternative approaches relying on expensive ultrasound probes or high-powered data processing.

According to a further aspect of the invention, there is provided a computer-implemented method of assessing ultrasound measurement data, comprising: providing a machine learning model trained using the method of training a machine learning model of any disclosed embodiment; receiving input data comprising a plurality of input frames of ultrasound measurement data, each input frame corresponding to a different imaging plane of ultrasound measurement data; and using the trained machine learning model to generate a quality metric for each of the input frames, the quality metric quantifying a relative capacity of the input frame to provide information about a respective one of the target anatomical features.

According to a further aspect of the invention, there is provided a computer-implemented method of assessing ultrasound measurement data, comprising: training a machine learning model using the method of training a machine learning model of any disclosed embodiment; receiving input data comprising a plurality of input frames of ultrasound measurement data, each input frame corresponding to a different imaging plane of ultrasound measurement data; and using the trained machine learning model to generate a quality metric for each of the input frames, the quality metric quantifying a relative capacity of the input frame to provide information about a respective one of the target anatomical features.

According to a further aspect of the invention, there is provided a method of determining information about an anatomical feature, comprising: performing ultrasound measurements on a subject to obtain a plurality of input frames of ultrasound measurement data, each input frame corresponding to a different imaging plane of ultrasound measurement data; and using a machine learning model trained using the method of training a machine learning model of any disclosed embodiment to generate a quality metric for each of the input frames, the quality metric quantifying a relative capacity of the input frame to provide information about a respective one of the target anatomical features.

According to a further aspect of the invention, there is provided an ultrasound system, comprising: an ultrasound probe; and a data processing system configured to perform the method of assessing ultrasound measurement data of any disclosed embodiment to assess ultrasound measurement data obtained by the ultrasound probe.

Embodiments of the disclosure will be further described by way of example only with reference to the accompanying drawings.

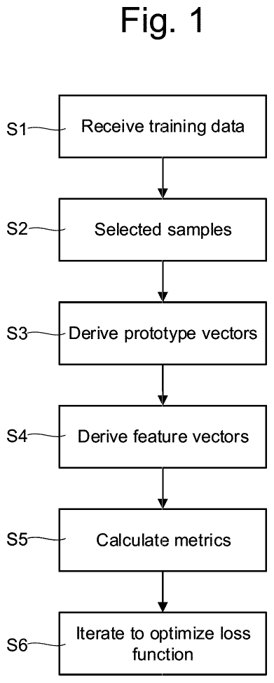

FIG. 1 is a flow chart depicting a framework for a method of training a machine learning model.

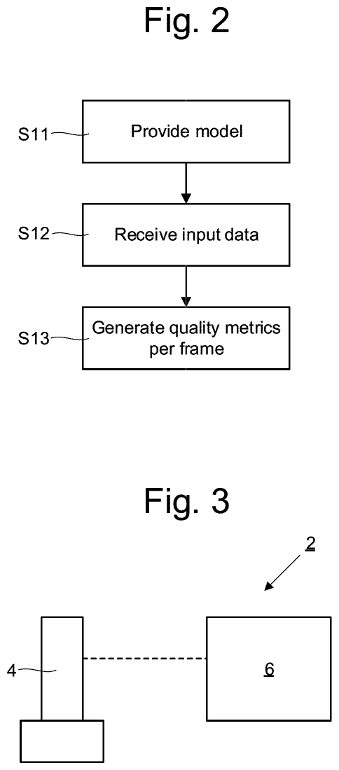

FIG. 2 is a flow chart depicting a framework for a method of assessing ultrasound measurement data.

FIG. 3 schematically depicts an ultrasound system.

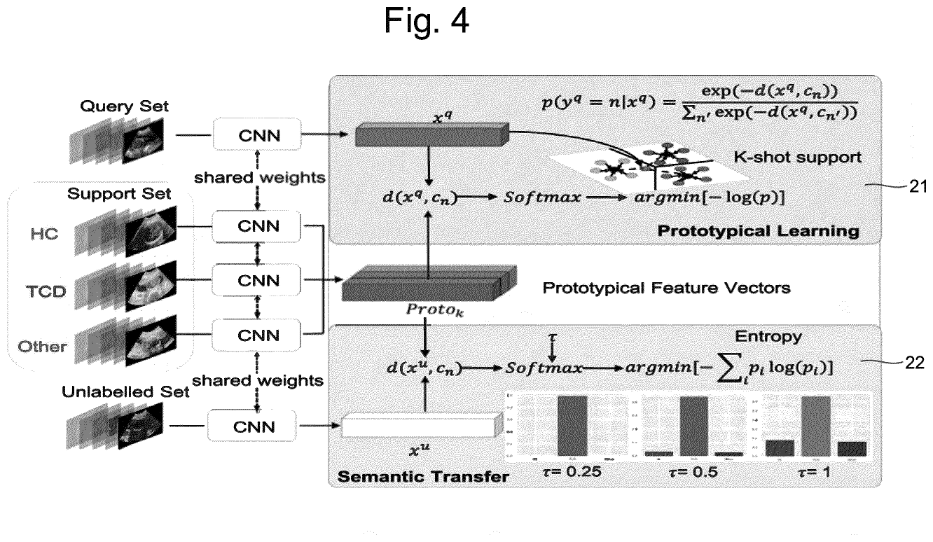

FIG. 4 schematically depicts a framework for training a machine learning model according to a detailed example.

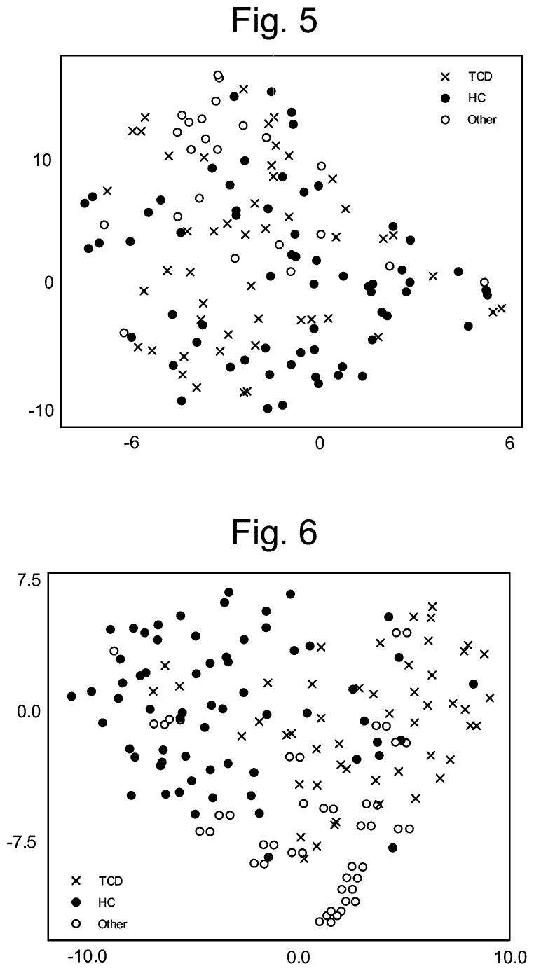

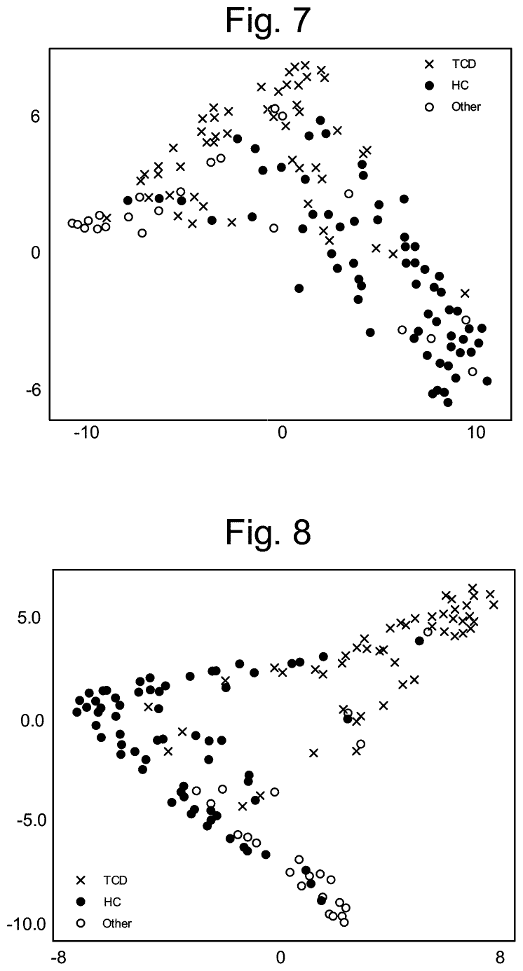

FIGS. 5-8 depict t-distributed stochastic neighbor embedding (t-SNE) embeddings of global features on test frames: FIG. 5 shows baseline (cross-entropy, CE); FIG. 6 shows baseline (cross-entropy training signal annealing, CE TSA); FIG. 7 shows joint learning (α=1, β=0.5); FIG. 8 shows joint learning (α=1, β=0.1).

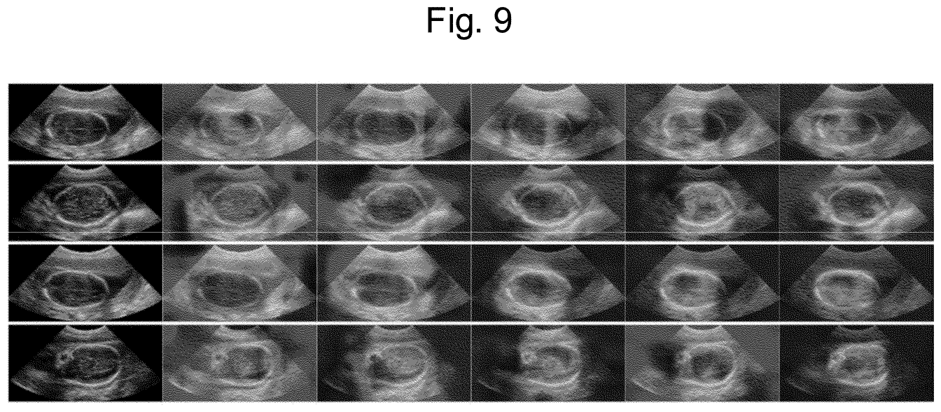

FIG. 9 depicts examples of TCD (Row 1 and 2) and HC (row 3 and 4) frames of ultrasound data. Column 1 depicts unprocessed frames. Columns 2-6 depict corresponding class activation maps (CAMs) obtained from: CE; CE TSA; joint learning with α=1, β=1; joint learning with α=1, β=0.5; and joint and learning with α=1, β=0.1, respectively.

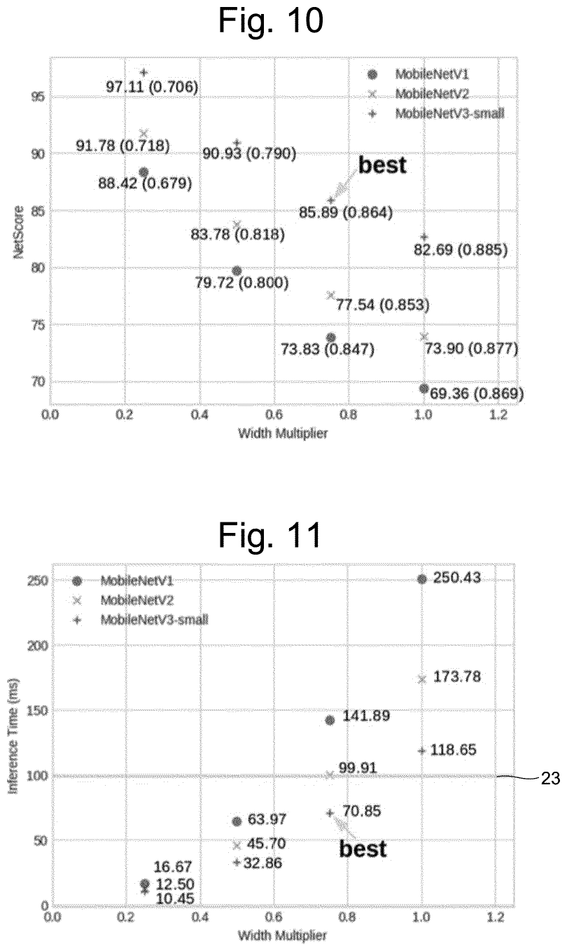

FIGS. 10 and 11 are graphs showing performance-model complexity trade-off. FIG. 10 shows NetScore (mAP). FIG. 11 shows inference time. All models were trained with joint learning configured with α=1, β=0.1.

Various embodiments of the disclosure relate to methods that are computer-implemented. Each step of the disclosed methods may be performed by a computer in the most general sense of the term, meaning any device capable of performing the data processing steps of the method, including dedicated digital circuits. The computer may comprise various combinations of known computer elements, including for example CPUs, RAM, SSDs, motherboards, network connections, firmware, software, and/or other elements known in the art that allow the computer to perform the required computing operations. The required computing operations may be defined by one or more computer programs. The one or more computer programs may be provided in the form of media or data carriers, optionally non-transitory media, storing computer readable instructions. When the computer readable instructions are read by the computer, the computer performs the required method steps. The computer may consist of a self-contained unit, such as a general-purpose desktop computer, laptop, tablet, mobile telephone, or other smart device. Alternatively, the computer may consist of a distributed computing system having plural different computers connected to each other via a network such as the internet or an intranet.

Embodiments of the disclosure concern training and use of a machine learning model that can improve ultrasound measurements by providing an automated assessment of the quality of ultrasound measurement data, in particular on an image plane by image plane basis.

### Training the Machine Learning Model

FIG. 1 is a flow chart depicting a framework for methods of training a machine learning model according to the present disclosure. A detailed example implementation is described further below in the section DETAILED EXAMPLE.

In step S1, the method comprises receiving training data. The training data comprises a plurality of classified frames of ultrasound measurement data. Each classified frame represents a single imaging plane. The classification of the classified frames indicates, for each of at least a subset of the classified frames, that the respective imaging plane is capable of providing information about a respective target anatomical feature (e.g. at a predetermined quality threshold or above). Each target anatomical feature may thus correspond to a respective one of the classified frame classes. The plurality of classified frames may comprise one or more classified frames that are classified according to a further class characteristic. The further class characteristic may be a characteristic other than being capable of providing information about a target anatomical feature (at a predetermined quality threshold or above). For example, the further class characteristic may be that the imaging plane represents background signal only (e.g. signal that is not capable of providing information about any target anatomic feature at a predetermined quality threshold or above). The classified frames may be referred to as labelled frames or labelled images, where the label represents the classification (e.g. which of the target anatomical features the classified frame can provide information about, or that the classified frames represent background only).

In some embodiments, the target anatomical features may include at least two target anatomical features. In some embodiments, the at least two target anatomical features comprise fetal head circumference (HC) and trans-cerebellar diameter (TCD). In such embodiments, the classified frames may correspond to imaging planes where information about HC or TCD is obtainable/visible. Various approaches may be used to quantify how suitable a given frame is for providing information about the target anatomical feature of interest. For example, a score may be calculated for each frame that represents how suitable that frame is for providing the information of interest. The frames may then be classified according to whether the score corresponding to a particular target anatomical feature is higher or lower than a predetermined threshold. For example, if a frame has a score that is higher than a respective predetermined threshold for TCD and lower than a respective predetermined threshold for HC, that frame can be classified as a TCD-suitable frame. If a frame has a score that is higher than the respective predetermined threshold for HC and lower than the respective predetermined threshold for TCD, that frame can be classified as a HC-suitable frame. If a frame has a score that is lower than the thresholds for all of the target anatomical features (e.g. HC and TCD), that frame may be classified as a background frame (i.e. not capable of providing information about any of the target anatomical features at a predetermined quality threshold or above).

In one embodiment, the following clinical criteria for scoring frames was used. Frames suitable for assessing TCD were scored from 1-9 based on the following factors: the relevant feature in the frame being horizontal scores 1; 30% or more magnification scores 1; symmetrical hemispheres scores 1; cavum septum pellucidum (CSP) (clear scores 2, suspected scores 1); Thalami (clear scores 2, suspected scores 1); cerebellar edge (clear scores 2, unclear scores 1). Frames suitable for assessing HC were scored from 1-9 based on the following factors: the relevant feature in the frame being horizontal scores 1; 30% or more magnification scores 1; symmetrical hemispheres scores 1; cavum septum pellucidum (CSP) (clear scores 2, suspected scores 1); Thalami (clear scores 2, suspected scores 1); No cerebellum visible scores 1; HC oval scores 1. In the detailed example described below, two experienced sonographers annotated three frames only per video (TCD, HC and background) and scored them. Frames having a score of 6 or more were deemed to be capable of providing information about the target anatomical feature of interest (TCD or HC) and classified accordingly. Frames having lower scores were classified as background.

In step S2, a selection is made from the classified frames received in step S1. The selection includes a first sample of frames and a second sample of frames. Each sample comprises multiple frames. The first sample of frames may be referred to as a support set. The second sample of frames may be referred to as a query set. The query set is typically significantly larger than the support set.

In step S3, a machine learning model is used to derive a prototype feature vector for each of one or more target anatomical features. Various machine learning models may be used in principle. In some embodiments, the machine learning model comprises a deep learning algorithm. The deep learning algorithm may comprise a convolutional neural network for example.

Each prototype feature vector may be derived as follows. Feature vectors are obtained by inputting to the machine learning model frames from the first sample that correspond to (i.e. belong to a classified frame class that corresponds to) a respective one of the target anatomical features. For example, in an embodiment where the target anatomical features comprise TCD and HC, a first prototype feature vector may be obtained for TCD as the target anatomical feature and a second prototype feature vector may be obtained for HC as the target anatomical feature. Each prototype feature vector may be obtained by averaging over feature vectors corresponding to the respective classified frame class. Thus, a prototype feature vector for a given target anatomical feature may be obtained by averaging over the feature vectors that correspond to that target anatomical feature. The first prototype feature vector may thus be obtained by averaging over feature vectors obtained for TCD as the target anatomical feature. The second prototype feature vector may be obtained by averaging over feature vectors obtained for HC as the target anatomical feature.

In some embodiments, step S3 further comprises deriving a prototype feature vector for a further class characteristic such as background signal only. The prototype feature vector for the further class characteristic is derived from feature vectors obtained by inputting to the machine learning model frames from the first sample that are classified as corresponding to the further class characteristic (e.g. background signal only).

More generally, prototype feature vectors can be derived for a wide range of class characteristics, including characteristics related to the type of imaging plane, the level of quality of the imaging plane, and others. A prototype feature vector in the context of the present disclosure encompasses any categorical representation in an embedding space. Typically, the prototype feature vector will be a high dimensional vector containing semantic information of a specific class.

In step S4, the machine learning model is used to derive a feature vector for each of the frames in the second sample.

In step S5, the method comprises calculating metrics representing respective distances (e.g. Euclidean distances), in an embedded space of the feature vectors, between each of the feature vectors derived in step S4 and each of the prototype feature vectors derived in step S3.

For example, in an embodiment where the target anatomical features comprise TCD and HC, metrics can be calculated that represent distances between each feature vector and a prototype feature vector representing TCD (derived in step S3) and distances between each feature vector and a prototype feature vector representing HC (derived in step S3).

In step S6, the method comprises iteratively modifying parameters of the machine learning model and repeating steps S2-S5 to optimize a loss function that is a function of the metrics calculated in step S5. Each of two or more of the repetitions may be performed with different first and/or second samples.

Various loss functions may be used. In some embodiments, as exemplified in the detailed example below, a probability distribution over all classes is calculated with a distance-based softmax. A cross-entropy loss term may then be calculated. Because this term depends on the metrics representing distances, this term may be referred to as a metric-based cross-entropy loss term L_{metric}. An example of this type of term is discussed below in the section “Prototypical Learning Module”. The loss function may thus include a first cross-entropy loss term that is a function of the metrics calculated in step S5 for feature vectors derived from the first and second samples selected in step S2. The loss function may be configured to introduce a constraint that data from the same class should be similar in the embedding space, for example by favouring lowering of distances in the embedded space, for each classified frame class (e.g. corresponding to a given target anatomical feature), between the prototype feature vector for the classified frame class and the feature vectors derived in step S4 from frames corresponding to the same classified frame class (e.g. the target anatomical feature to which the prototype feature vector corresponds).

In some embodiments, step S1 further comprises receiving a plurality of unclassified frames of ultrasound measurement data. The unclassified frames may be referred to as unlabelled frames or unlabelled images. In such embodiments, step S2 may further comprise selecting from the plurality of unclassified frames a third sample of frames. The third sample of frames may be referred to as an unlabelled set. Step S4 may then further comprise using the machine learning model to derive a feature vector for each of the frames in the third sample.

In an embodiment, the loss function used in step S6 may include a second loss term that is a function of the metrics calculated in step S5 for feature vectors derived from the third sample. The second loss term may comprise a temperature-tuned entropy loss that causes transfer of information from the prototype feature vectors derived in step S3 to unlabelled datapoints by minimizing the entropy of a temperature tuned softmax. The second loss term may be referred to as a semantic transfer loss term L_{ST}. An example of this type of term is discussed below in the section “Unsupervised Semantic Transfer”.

In an embodiment, the machine learning model comprises a further fully-connected layer that maps the feature vectors of the frames in the second sample to class scores directly. This serves as an auxiliary classifier and may be trained with a cross-entropy loss to provide a further cross-entropy loss term, L_{CE}, in the loss function using in step S5. In an embodiment, training signal annealing (TSA) is introduced to the further cross-entropy loss term, L_{CE}, as exemplified in further detail below.

### Application of Trained Model

FIG. 2 is a flow chart depicting a framework for a method of assessing ultrasound measurement data.

In step S11, the method comprises providing a machine learning model trained using any of the methods for training a machine learning model described herein (e.g. with reference to FIG. 1 or FIG. 4). The providing of the machine learning model may include the training of the machine learning model.

In step S12, input data comprising a plurality of input frames of ultrasound measurement data is received. Each input frame represents a different imaging plane of ultrasound measurement data.

In step S13, the trained machine learning model generates a quality metric for each of the input frames. The quality metric quantifies a relative capacity of the input frame to provide information about a respective one of the target anatomical features. The quality metric may be calculated by using the trained machine learning model to calculate a probability that a feature vector corresponding to a respective input frame belongs to a particular class of feature vectors represented by the model. A probability distribution for each of the classes over an embedded space may, for example, be used to determine the probability that the feature vector belongs to each of the available classes (e.g. target anatomical features). If the probability is very high for a first of the classes and low for all of the other classes, it may be concluded that the quality metric for the input frame should be high in respect of the first class (and low in respect of all of the other classes).

In some embodiments, the trained machine-learning model is used to generate a plurality of quality metrics for each input frame. Each quality metric may quantify a relative capacity of the input frame to provide information about a different respective one of the target anatomical features. For example, a quality metric for HC may be calculated for the input frame and a quality metric for TCD may be calculated for the input frame. The quality metrics are calculated for each input frame by calculating a feature vector corresponding to the input frame and comparing the calculated feature vector with a probability distribution over the embedded space for each target anatomic feature (e.g. with a probability distribution for HC and a probability distribution of TCD).

The generated quality metrics may be used as the basis for selecting a suitable frame for further processing. For example, an input frame may be selected on the basis of a generated quality metric (e.g. whether a quality metric of interest is high). The selected input frame may then be processed to determine information about the target anatomical feature corresponding the generated quality metric that was used to perform the selection. For example, when a quality metric for HC is high, a corresponding input frame may be processed to obtain information about HC. Alternatively, a distribution of quality metrics may be used to decide whether or not a sequence of video contains any frames of adequate quality to obtain the required information. If none of the frames are good enough the video should be taken again.

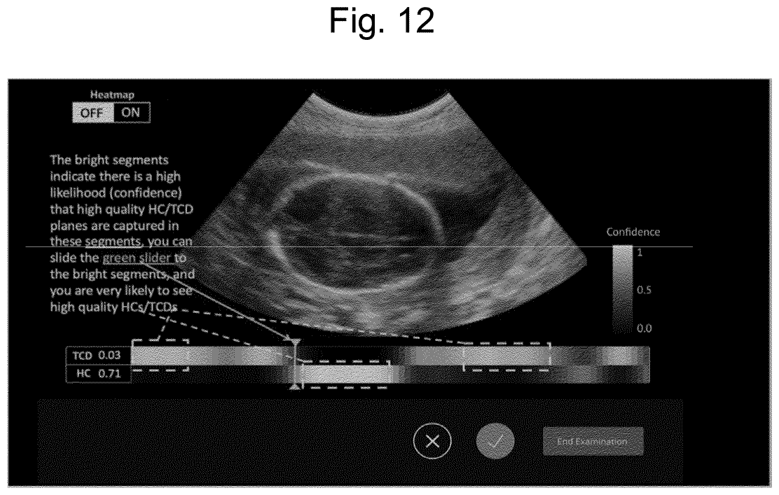

Embodiments of the disclosure thus allow feedback to be obtained about a relevant quality of ultrasound imaging data while a video is being taken. The feedback is based on automatically obtained quality metrics and may assist a user to decide whether to accept or retake the video and/or how to select the most useful frames in the video. The quality assessment may run through each acquired video frame by frame. An example screen shot from an example implementation is shown in FIG. 12. Here, the horizontal “colorbars” shown at the bottom of the screen depict a temporally smoothed labelling confidence score over a full 8 seconds of video. The confidence score is a numerical value between 0 and 1 and is an example of a quality metric. The confidence score (quality metric) is mapped to a greylevel scale in the example shown. The brighter the shade of grey (white is brightest) the greater likelihood that a target plane has a high capacity to obtain the desired information about the target anatomical feature of interest. In this realisation, the target anatomical features of interest are the HC for measuring the fetal head circumference and TCD for measuring the transcerebellar diameter, so there are two horizontal bars. It can be seen in this example that frames that are suitable for HC are generally not suitable for TCD and vice versa. Users can zoom into a particular section of the colorbars and see the corresponding segment of video. The user can also choose to display a heatmap or not, which depicts and highlights the structures that are highly correlated with the decision making of the machine learning model. This acts as a visual confidence measure system and the confidence numerical value (e.g. quality metric) can also be shown on the screen to allow for cross-checking.

### Ultrasound System

FIG. 3 schematically depicts an ultrasound system 2. The system 2 comprises an ultrasound probe 4 and a data processing system 6. The ultrasound probe 4 may be linked to the data processing system 6 by a data connection capable of transferring data between the entities. The data connection may be wired or wireless. In some embodiments, the data processing system 6 may be configured to perform any of the methods of assessing ultrasound measurement data disclosed herein, for example as discussed above with reference to FIG. 2, using as input ultrasound measurement data obtained by the ultrasound probe 4.

### DETAILED EXAMPLE

### Machine Learning Framework

FIG. 4 depicts a framework for training a machine learning model according to a detailed example. In this example, the machine learning model comprises a Convolutional Neural Network (CNN), a prototypical learning module 21 (e.g. configured to derive the prototype feature vectors in step S3 of FIG. 1), and a semantic transfer module 22. The learning task is formulated as a multi-way classification problem and the learned model is employed to automatically label unseen video frames (also referred to as input frames) in a dense manner (e.g. generate a quality metric quantifying a relative capacity of each input frame to provide information about a respective one of the target anatomical features).

For each training iteration, we randomly sample a query set X^{Q} (a small batch of labelled images, referred to above as a “first sample of frames” in the discussion referring to FIG. 1) and a small support set X^{S} (few images per class, smaller than query set, referred to above as a “second sample of frames” in the discussion referred to FIG. 1) from n labelled frames (e.g. the classified frames received in step S1 of FIG. 1), and an unlabelled set X^{U} (referred to above as a “third sample of frames”) from m unlabelled frames (n<<m) (e.g. the unclassified frames received in step S1 of FIG. 1).

### CNN Architecture

In this example, MobileNet (Howard, A. G., et al.: MobileNets: efficient convolutional neural networks for mobile vision applications. arXiv preprint arXiv: 1704.04861 (2017)) introducing depth-wise separable convolution was used to reduce the computational cost and the number of trainable parameters of convolution layers (other architectures might also be used). This is achieved by applying a channel-wise convolution first followed by 1×1 point-wise convolution to linearly combine the feature maps across the channels. The complete CNN architecture is summarized in Table.1 that consists of 30 layers (14 convolutional layers, 13 depth-wise convolutional layers, 1 global average pooling layers, 1 fully connected layer and 1 softmax classifier). Each video frame is fed through the CNN and outputs a 7×7 feature map. A global pooling operation is applied to pool the feature maps channel wise (1024 feature channels) to have a 1024-dimensional feature vector. Class scores are obtained by a fully connection operation that maps the feature vector to class scores and then a softmax is used to generate a probability distribution across classes.

TABLE 1
CNN architecture. Conv/s2: 2D convolution with a stride of
2 pixels; Conv dw/s1: 2D depth-wise convolution with a stride
of 1 pixel; Avg Pool/s1: Global average pooling with a stride
of 1 pixel.; FC/s1: Fully connection with a stride of 1.
Softmax/s1: generate class-wise probability.

| Type/Stride | Filter Shape | Input Size |
|---|---|---|
| Conv2/s2 | 3 × 3 × 3 × 32 | 224 × 224 × 3 |
| Conv dw/s1 | 3 × 3 × 32 dw | 112 × 112 × 32 |
| Conv/s1 | 1 × 1 × 32 × 64 | 112 × 112 × 32 |
| Conv dw/s2 | 3 × 3 × 64 dw | 112 × 112 × 64 |
| Conv/s1 | 1 × 1 × 64 × 128 | 56 × 56 × 64 |
| Conv dw/s1 | 3 × 3 × 128 dw | 56 × 56 × 128 |
| Conv/s1 | 1 × 1 × 128 × 128 | 56 × 56 × 128 |
| Conv dw/s2 | 3 × 3 × 128 dw | 56 × 56 × 128 |
| Conv/s1 | 1 × 1 × 128 × 256 | 28 × 28 × 128 |
| Conv dw/s1 | 3 × 3 × 256 dw | 28 × 28 × 256 |
| Conv/s1 | 1 × 1 × 256 × 256 | 28 × 28 × 256 |
| Conv dw/s2 | 3 × 3 × 256 dw | 28 × 28 × 256 |
| Conv/s1 | 1 × 1 × 256 × 512 | 14 × 14 × 256 |
| 5 × Conv dw/s1 | 3 × 3 × 512 dw | 14 × 14 × 512 |
| Conv/s1 | 1 × 1 × 512 × 512 | 14 × 14 × 512 |
| Conv dw/s2 | 3 × 3 × 512 dw | 14 × 14 × 512 |
| Conv/s1 | 1 × 1 × 512 × 1024 | 7 × 7 × 512 |
| Conv dw/s2 | 3 × 3 × 1024 dw | 7 × 7 × 1024 |
| Conv/s1 | 1 × 1 × 1024 × 1024 | 7 × 7 × 1024 |
| Avg Pool /s1 | Pool 7 × 7 | 7 × 7 × 1024 |
| FC/s1 | 1024 × 1000 | 1 × 1 × 1024 |
| Softmax/s1 | Classifier | 1 × 1 × 1000 |

### Prototypical Learning Module

To avoid overfitting, a prototypical learning module 21 may be introduced as illustrated in FIG. 4. Few-shot examples (a support set) are sampled from each class and fed through a CNN f_{θ} first to compute categorical prototypes. Given a support set of N_{S} frames x_{S}∈X^{S}^{ₖ} from the k^{th} class, prototype Protoₖ is computed as the mean of the embedded D-dimensional feature vectors f_{θ}(x_{S})∈ [symbol] ^{D}:

$${Proto}_{k} = \frac{1}{{N}_{S}} {\sum }_{{x}_{s} ϵ {X}^{S} k} {f}_{θ} ( {x}_{S} ) \qquad ( 1 )$$

Then, a query set of N_{Q} labelled frames (larger than the support set) is sampled. For each query point (x_{q},y_{q}), where x_{q}∈X^{Q} and y_{q}∈{1, . . . ,K} (K=3 in this example), the Euclidean distance to each prototype is measured and a probability distribution over all classes is produced with a distance based softmax. The metric based cross-entropy loss is then calculated:

$${L}_{metric} ( {X}^{S} , {X}^{Q} ) = - \frac{1}{{N}_{Q}} {\sum }_{\{ {x}_{q} , {y}_{q} \} ϵ {X}^{Q}} log \frac{exp ( - {‖ {f}_{θ} ( {x}_{q} ) - {Proto}_{{y}_{q}} ‖}^{2} )}{{\sum }_{k = 1}^{K} exp ( - {‖ {f}_{θ} ( {x}_{q} ) - {Proto}_{{y}_{q}} ‖}^{2} )} \qquad ( 2 )$$

With the loss, the constraint that data from the same class should be similar in the embedding space is introduced. Also, it is found that this loss can provide a guidance to stabilize the unsupervised semantic transfer.

### Unsupervised Semantic Transfer

A semantic transfer objective, L_{ST}(X^{U}) is defined that transfers information from the prototypes produced above to unlabelled datapoints by minimizing the entropy of a temperature-tuned softmax. Entropy minimization can be used for unsupervised and semi-supervised learning by encouraging low density separation between classes. At each training epoch, an unlabelled set X^{U} of N_{U} frames (N_{U}=N_{Q}) is sampled, then for each datapoint in the unlabelled set xᵤ∈X^{U} the Euclidean distance between its feature embedding and each categorical prototype is again computed. The semantic transfer loss is then defined as:

$${L}_{ST} ( {X}^{U} ) = - \frac{1}{{N}_{U}} {\sum }_{{x}_{u} ϵ {X}^{U}} {\sum }_{k ϵ \{ 1 ... K \}} {τ}^{- 1} P ( {x}_{u} , {Proto}_{k} ) log {τ}^{- 1} {P}_{τ} ( {x}_{u} , {Proto}_{k} ) \qquad ( 3 )$$

Where P(xᵤ,Protoₖ) is a softmax function that generates a probability distribution over all classes based on: −∥f_{θ}(xᵤ)−Protoₖ∥²k and τ is the temperature of the softmax. The softmax temperature can then be tuned. As shown in FIG. 4, the distribution becomes one-hot when the temperature approaches 0, whereas it becomes more uniformly distributed when increasing the temperature. Intuitively, a small temperature encourages each unlabelled frame to be very similar to one class, whereas a larger temperature will allow it to be similar to multiple classes.

### Joint Learning:

In addition to the above, in the present example another fully-connected layer is introduced that maps the feature vectors of query frames to class scores directly. This serves as an auxiliary classifier, trained with a cross-entropy (CE) loss, making it possible to investigate the interactions between direct learning and metric learning. To mitigate overfitting, in one realisation training signal annealing (TSA) (e.g. as described in Xie, Q., Dai, Z., Hovy, E., Luong, M. T., Le, Q. V.: Unsupervised data augmentation for consistency training. arXiv preprint arXiv:1904.12848 (2019)) is introduced to the cross-entropy loss:

$${L}_{CE} ( {X}^{Q} ) = - \frac{1}{{N}_{Q}} {\sum }_{\{ {x}_{q} , {y}_{q} \} ϵ {X}^{Q}} [ - I \{ {P}_{θ} ( {y}_{q} \vert {x}_{q} ) < {η}_{t} \} ] log {P}_{θ} ( {y}_{q} \vert {x}_{q} ) \qquad ( 4 )$$

Where I{·} is the indicator function and P_{θ}(y_{q}|x_{q}) is the probability of x_{q} belonging to the class y_{q}. Specifically, the example (x_{q},y_{q}) does not contribute to the loss function if the model predicted probability surpasses a threshold ηₜ, at training step t. We set ηₜ=exp

$$( ( \frac{t}{T} - 1 ) * 5 ) * ( 1 - \frac{1}{K} ) + \frac{1}{K}$$

that corresponds to the exponential schedule in Xie et al. realising most of the supervised signal at the end of training. Intuitively, this is to prevent the model from overfitting too quickly by penalizing over confident prediction in the early stage of training. Finally, the model jointly optimizes over the objective function as follows:

L(X^{S},X^{Q},X^{U})=L_{ST}(X^{U})+αL_{metric}(X^{S},X^{Q})+βL_{CE}(X^{Q}) (5)

where the hyperparameters α and β determine the influence of the metric learning and the direct learning, respectively.

### Experiments Results:

Evaluation of the models was performed using Average Precision (AP) measured on frame level labels. For each class, a correct detection is counted if it is a positive prediction with confidence above a certain threshold (ranging from 0.1 to 0.9). AP is reported for the individual classes as well as mean Average Precision (mAP) in Table 2. It was found that directly learning with CE loss can result in poor generalization to test data as indicated by the baseline model (CE), which is the worst among all models. Note that there is an improvement over all metrics after applying TSA to the CE loss but it is marginal. Moreover, it was found that L metric (Eq.2) can significantly improve model generalization. When applying a full metric learning signal (i.e. fixing α=1), all metrics increase when reducing the contribution of the cross-entropy loss (i.e. reducing β) (Eq. 5).

TABLE 2
Performance measures over different learning configurations on the testset
(CNN Backbone shown Tab. 1, Width multiplier: 1).

| | Models | | | | | | | | |
|---|---|---|---|---|---|---|---|---|---|
| | Baselines | | α:β | α = 1 | | | β = 1 | | |
| Categories | CE | CE_{TSA} | 1:1 | β = 0.5 | β = 0.1 | β = 0 | α = 0.5 | α = 0.1 | α = 0 |
| TCD | 0.459 | 0.547 | 0.573 | 0.735 | 0.746 | 0.740 | 0.639 | 0.614 | 0.611 |
| HC | 0.595 | 0.643 | 0.692 | 0.859 | 0.897 | 0.889 | 0.784 | 0.754 | 0.742 |
| mAP | 0.525 | 0.618 | 0.678 | 0.834 | 0.869 | 0.837 | 0.783 | 0.729 | 0.713 |

The best performance is achieved when β equals to 0.1, whereas, when applying a full cross-entropy signal (i.e. fixing β=1), the performance of models overall is less superior than for models trained with a full metric learning signal. Of note, all metrics drop in value when gradually reducing the contribution of the metric learning loss from 0.5 to 0.1 to 0. It was also found that, overall, there is a higher AP for the HC than TCD and this may be because some low quality TCD frames (with unclear cerebellar edges) are prone to be confused as a HC frame. t-SNE was also performed on the test dataset, as summarized in FIGS. 5-8: feature embedding (feature vectors after global pooling) visualisation. It was found that the baseline model (CE) is severely overfitted as depicted in FIG. 5, that categorical clusters are not formed at all and datapoints are distributed randomly. Of note that in FIG. 6 a separation between classes can be seen after applying TSA to the CE loss. However, there is still no clear separation between the classes and all datapoints are still clustered together. In contrast, categorical clusters in FIGS. 7 and 8 are formed well with clear separation between the classes with the joint learning. We also observe that the stronger the metric learning signal, the stronger the “forces” that push the clusters away from each other.

In the heat maps of FIG. 9, we give examples of HC and TCD clips that have been identified by different models and class activation mappings. We linearly combine the feature maps before global pooling with score mapping weights to produce frame-wise class activation maps. We found that CAMs produced by CE and CETSA are extremely random indicating that the models do not learn where the discriminative features are. However, the joint learning models produce more discriminative CAMs and learn to highlight a wide variety of different anatomical structures, such as the skull, cerebellum, thalami and CSP.

To evaluate models' on-device performance, as shown in FIGS. 10 and 11, we estimate the NetScore metric Ω=20 log₁₀ (mAP^{γ}p^{-δ}c^{-ϵ}) (Wong, A.: NetScore: towards universal metrics for large-scale performance analysis of deep neural networks for practical usage. arXiv:1806.05512 (2018)), which measures model efficiency as a trade-off between mean average precision (mAP, percent), number of trainable parameters (p, millions) and number of float operations (FLOPs, billions). We construct smaller and less computationally expensive models by a width multiplier. Of note, although mAP drops continuously when networks become thinner, higher netscores are achieved because models become more compact and efficient. Moreover, we also report inference times, as shown in FIG. 11, for a single frame on the Huawei MediaPad equipped with KIRIN659 SoC. We found the thinner the networks, the faster the inference they achieved. As our videos are recorded at a rate of 10 frames/seconds (100 ms/frame), the models below the horizontal line 23 in FIG. 11 achieve real time inference. Considering jointly the NetScore, mAP and speed, the MobileNetV3-small with a width multiplier 0.75 (as indicated by the arrows) is the best and achieves comparable mAP (0.864) to a full capacity MobileNetV1 (0.869) but more than triple the inference speed (70.85 ms compared to 250.43 ms).

1. A computer-implemented method of training a machine learning model to assess ultrasound measurement data, the method comprising:
(a) receiving training data comprising a plurality of classified frames of ultrasound measurement data, each of at least a subset of the classified frames being classified as representing an imaging plane capable of providing information about a respective target anatomical feature corresponding to the classified frame class;
(b) selecting from the plurality of classified frames a first sample of frames and a second sample of frames;
(c) using the machine learning model to derive a prototype feature vector for each of one or more target anatomical features, each prototype feature vector being derived from feature vectors obtained by inputting to the machine learning model frames from the first sample that belong to a classified frame class corresponding to a respective one of the target anatomical features;
(d) using the machine learning model to derive a feature vector for each of the frames in the second sample;
(e) calculating metrics representing respective distances, in an embedded space of the feature vectors, between each of the feature vectors derived in (d) and each of the prototype feature vectors derived in (c); and
(f) iteratively modifying parameters of the machine learning model and repeating (b)-(e) to optimize a loss function that is a function of the metrics calculated in (e).

2. The method of claim 1, wherein the one or more target anatomical features comprises at least two target anatomical features.

3. The method of claim 2, wherein the target anatomical features include one or more of the following: fetal head circumference, HC; trans-cerebellar diameter, TCD.

4. The method of claim 1, wherein each metric comprises a Euclidean distance between the respective feature vector and prototype feature vector.

5. The method of claim 1, wherein the loss function includes a first cross-entropy loss term that is a function of the metrics calculated in (e) for feature vectors derived from the first and second samples.

6. The method of claim 1, wherein each prototype feature vector is obtained in (c) by averaging over feature vectors corresponding to the respective classified frame class.

7. The method of claim 1, wherein the loss function is configured to favour lowering of the distances in the embedded space, for each classified frame class, between the prototype feature vector for the classified frame class and the feature vectors derived in (d) from frames corresponding to the same classified frame class.

8. The method of claim 1, wherein the repeating of (b)-(e) in (f) is performed with different first and/or second samples in each of two or more of the iterations.

9. The method of claim 1, wherein:
the step (a) further comprises receiving a plurality of unclassified frames of ultrasound measurement data;
the step (b) further comprises selecting from the plurality of unclassified frames a third sample of frames; and
the step (d) further comprises using the machine learning model to derive a feature vector for each of the frames in the third sample.

10. The method of claim 9, wherein the loss function includes a second loss term that is a function of the metrics calculated in (e) for feature vectors derived from the third sample.

11. The method of claim 10, wherein the second loss term comprises a temperature-tuned entropy loss term.

12. The method of claim 1, wherein the plurality of classified frames comprises one or more classified frames that are classified according to a further class characteristic, the further class characteristic being a characteristic other than being capable of providing information about a target anatomical feature.

13. The method of claim 12, wherein step (c) additionally comprises deriving a prototype feature vector for the further class characteristic, the prototype feature vector being derived from feature vectors obtained by inputting to the machine learning model frames from the first sample that are classified as corresponding to the further class characteristic.

14. The method of claim 13, wherein the further class characteristic is that the imaging plane represents background signal that is not capable of providing information about a target anatomic feature corresponding to any other prototype feature vector at a predetermined quality threshold or above.

15. The method of claim 1, wherein the machine learning model comprises a deep learning algorithm.

16. The method of claim 15, wherein the deep learning algorithm comprises a convolutional neural network.

17. A computer-implemented method of assessing ultrasound measurement data, comprising:
providing a machine learning model trained using the method of claim 1;
receiving input data comprising a plurality of input frames of ultrasound measurement data, each input frame corresponding to a different imaging plane of ultrasound measurement data; and
using the trained machine learning model to generate a quality metric for each of the input frames, the quality metric quantifying a relative capacity of the input frame to provide information about a respective one of the target anatomical features.

18. A computer-implemented method of assessing ultrasound measurement data, comprising:
training a machine learning model using the method of claim 1;
receiving input data comprising a plurality of input frames of ultrasound measurement data, each input frame corresponding to a different imaging plane of ultrasound measurement data; and
using the trained machine learning model to generate a quality metric for each of the input frames, the quality metric quantifying a relative capacity of the input frame to provide information about a respective one of the target anatomical features.

19. The method of claim 17, wherein the trained machine learning model is used to generate a plurality of quality metrics for each input frame, each quality metric quantifying a relative capacity of the input frame to provide information about a different respective one of the target anatomical features.

20. The method of claim 17, wherein the quality metrics are calculated for each input frame by calculating a feature vector corresponding to the input frame and comparing the calculated feature vector with a probability distribution over the embedded space for each target anatomic feature.

21. The method of claim 17, further comprising selecting an input frame based on a generated quality metric and using the selected input frame to determine information about the target anatomical feature corresponding to the generated quality metric.

22. (canceled)

23. A computer-readable medium comprising instructions which, when executed by a computer, cause the computer to carry out the method of claim 1.

24. A method of determining information about an anatomical feature, comprising:
performing ultrasound measurements on a subject to obtain a plurality of input frames of ultrasound measurement data, each input frame corresponding to a different imaging plane of ultrasound measurement data; and
using a machine learning model trained using the method of claim 1 to generate a quality metric for each of the input frames, the quality metric quantifying a relative capacity of the input frame to provide information about a respective one of the target anatomical features.

25. An ultrasound system, comprising:
an ultrasound probe; and
a data processing system configured to perform the method of claim 1 to assess ultrasound measurement data obtained by the ultrasound probe.
